(19) United States
(12) Patent Application Publication
(10) Pub. No.: US 2025/0354149 A1
(43) Pub. Date: Nov. 20, 2025

(54) L-RNA APTAMER-ANTISENSE OLIGONUCLEOTIDE CONJUGATES AND USES THEREOF

(71) Applicant: CITY UNIVERSITY OF HONG KONG, Kowloon (HK)

(72) Inventors: Chun Kit KWOK, Kowloon (HK); Haizhou ZHAO, Kowloon (HK)

(21) Appl. No.: 19/212,205

(22) Filed: May 19, 2025

Related U.S. Application Data
(60) Provisional application No. 63/649,850, filed on May 20, 2024.

(51) Int. Cl. C12N 15/113

(52) U.S. Cl. CPC C12N 15/113; C12N 2310/11; C12N 2310/16; C12N 2310/3519

(57) ABSTRACT

An L-form ribonucleic acid (L-RNA) aptamer-antisense oligonucleotide (ASO) conjugate comprise the L-RNA aptamer, which comprises a ribonucleic acid sequence. The ASO comprises a deoxyribonucleic acid sequence selected from a group. A method of imaging amyloid precursor protein (APP) rG4 in a cell comprising transfecting the cell with a messenger RNA of APP, permeating the cell, contacting the permeated cell with a cyanine3 (Cy3) labeled APP nucleic acid probe and the L-RNA aptamer-ASO conjugate, and subjecting the product to fluorescence microscopy analysis to produce an image of the APP rG4 in the cell.

### CROSS REFERENCE TO RELATED APPLICATIONS

This application relates to and claims the benefits of U.S. Provisional Application No. 63/649,850 filed May 20, 2024, the content of which is incorporated herein by reference in its entirety.

### REFERENCE TO A SEQUENCE LISTING

The present application is being filed along with a Sequence Listing in an electronic format. The Sequence Listing is provided as a file entitled “HP0354US_SeqList”, created Apr. 17, 2025, which is 42 KB in size. The information in the electronic format of the Sequence Listing is incorporated herein by reference in its entirety.

### BACKGROUND OF THE INVENTION

### 1. FIELD OF THE INVENTION

The present disclosure in general relates to a novel conjugate that recognizes and binds to G-quadruplexes (G4s) structure of a target nucleic acid. More particularly, the present disclosure relates to L-aptamer-antisense oligonucleotide (ASO) conjugate, which recognizes and binds to G4 structure of an amyloid precursor protein (APP) gene.

### 2. DESCRIPTION OF RELATED ART

Guanine (G)-rich sequences of single-stranded DNA and RNA can fold into stable, intra-or intermolecular secondary structures called G-quadruplexes (dG4s and rG4s). Four guanines interact with each other by Hoogsteen-hydrogen bonds to form a planar structure, G-quartet. Stacking of two or more G-quartets, connected by loop nucleotides forms a G4 structure, and it is further stabilized by monovalent cations (K⁺>Na⁺>Li⁺). Over the years, rG4s have been reported to have key roles in gene regulation and cellular processes, such as transcription, RNA splicing, translation, RNA stability, RNA localization and others. In addition, new studies have associated rG4s with diseases and cancers, making them one of the promising therapeutic targets for drug development.

G4 targeting is a topic of emerging interest, and since the first report of G4-specific chemical in 1997, more than hundreds of G4 ligands have been developed. Currently, major approaches employed for G4 targeting include the development of G4-specific chemicals, peptides and antibodies. The generation and application of these G4 tools have greatly promoted the understanding of G4 structure and biology, and the elucidation of the three-dimensional (3D) high resolution structure of the binding complexes provided fundamental insights on the future enhancement of these G4 tools. Despite the significant progresses made, the selective targeting of G4 of interest is still challenging due to the structural similarity of G4s, with only limited success so far.

In view of the foregoing, there is a continue interest in developing a novel tool for selectively recognizing and binding to rG4 structure in a target gene (e.g., the APP gene).

### SUMMARY

As embodied and broadly described herein, one aspect of the present disclosure is directed to an L-form ribonucleic acid (L-RNA) aptamer-antisense oligonucleotide (ASO) conjugate that recognizes and binds to amyloid precursor protein (APP) 3′-untranslated region (UTR) rG4 structure. The L-RNA aptamer-ASO conjugate has the structure of formula (I),

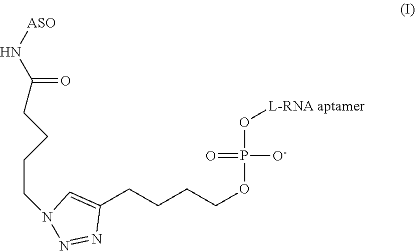

wherein,
the L-RNA aptamer comprises a ribonucleic acid sequence of SEQ ID NO: 1; and
the ASO comprises a deoxyribonucleic acid sequence selected from the group consisting of SEQ ID NOs: 2, 3 or 4.

According to optional embodiments of the present disclosure, the L-RNA aptamer-ASO conjugate further includes a fluorescein molecular disposed at the 5′-end of the ASO.

Another aspect of the present disclosure aims at providing a method of imaging APP rG4 in a cell. The method includes steps of:
(a) transfecting the cell with a messenger RNA of APP;
(b) permeating the cell,
(c) contacting the permeated cell of step (b) with a cyanine3 (Cy3) labeled APP nucleic acid probe and the present L-RNA aptamer-ASO conjugate having a fluorescein molecular disposed at the 5′-end of the ASO; and
(d) subjecting the product of step (c) to fluorescence microscopy analysis to produce an image of the APP rG4 in the cell.

According to embodiments of the present disclosure, in step (b), the cell is permeated by a non-ionic surfactant. Exemplary non-ionic surfactants suitable for use in the present method include polyethylene glycol octyl phenyl ether, and the like.

A further aspect of the present disclosure aims at providing a method of suppressing the expression of APP in a cell. The method includes transfecting the cell with a sufficient amount of the present L-RNA aptamer-ASO conjugate.

According to embodiments of the present disclosure, the transfection of the cell is achieved by use of lipofectamine.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 Synthesis of L-Apt.4-1c-ASO conjugate by click reaction. Lane 1: L-Apt.4-1c_5′Hexynyl, Lane 2: ASO DNA-3′Azide, Lane 3: ASO DNA-3′Azide reacted with L-Apt.4-1c_5′Hexynyl by click reaction to generate L-Apt.4-1c-ASO conjugate. Lane 4: The gel and column purified L-Apt.4-1c-ASO conjugate. The gel was stained by SYBR Gold.

FIG. 2 L-Apt.4-1c-ASO15nt_{(APP)} conjugate displays strong binding affinity and specificity toward the APP rG4 wt region over other non-targets. Strong binding was observed between L-Apt.4-1c-ASO15nt_{(APP)} and APP rG4 wt region. Weak binding was observed between L-Apt.4-1c-ASO15nt_{(APP)} with Bcl2 rG4 region, TRF2 rG4 region, MT3-MMP rG4 region. No binding was observed between L-Apt.4-1c-ASO15nt_{(APP)} with dG4 regions, such as Bcl2 dG4 region, TRF2 dG4 region, MT3-MMP dG4 region, hTERC (dG4 region, VEGF dG4 region, c-Kit 1 dG4 region, Bcl2Mid dG4 region, hTELO dG4 region. No binding was observed between L-Apt.4-1c-ASO15nt_{(APP)} with non-G4 targets, including DNA and RNA hairpin, single-stranded poly rA/rC/rU RNAs. The L-Apt.4-1c-ASO15nt_{(APP)} preferentially interact with APP rG4 wt region over other constructs tested.

FIG. 3A Different concentration (0, 30, 60 and 90 nM) effect of L-Apt.4-1c-ASO15nt_{(APP)} addition on APP expression in Hela cells. The APP expression decreased with increasing L-Apt.4-1c-ASO15nt_{(APP)} concentration. GAPDH was used as loading control. Cells were treated for 22 h.

FIG. 3B Normalized APP expression results from western blotting obtained from FIG. 3A. Results were obtained from three replicates. Error bars represented the standard error of mean. *, P<0.05.

FIG. 3C Different time point (4, 10, and 22 h) effect of L-Apt.4-1c-ASO15nt_{(APP)} addition on APP expression in Hela cells. The APP expression decreased with increasing time treatment. The concentration of L-Apt.4-1c-ASO15nt_{(APP)} was 60 nM.

FIG. 3D Normalized APP expression results from western blotting obtained from FIG. 3C. Results were obtained from three replicates. Error bars represented the standard error of mean. *, P<0.05.

FIG. 3E Comparison between L-Apt.4-1c-ASO15nt_{(APP)} and L-Apt.4-1c_5′Hexynyl. The APP expression both decreased, with L-Apt.4-1c-ASO15nt_{(APP)} having a stronger inhibitory effect.

FIG. 3F Normalized APP expression results from western blotting obtained from FIG. 3E. Results were obtained from three replicates. Error bars represented the standard error of mean. *, P<0.05.

FIG. 3G RNase H treatment assay detected by denaturing gel. RNase H cleaved the conjugate-APP rG4 region complex.

FIG. 4A DHX36 unwound APP rG4 in the presence of ATP, while not in the ATP non-hydrolysable analog, AMP-PNP. The ssRNA trap hybridized with the unwound APP rG4, showing duplex shift bands. M: ssRNA trap was boiled together with APP rG4 sequence to form duplex as shift marker in the native gel.

FIG. 4B L-Apt.4-1c-ASO15nt_{(APP)} conjugate effectively disrupts the interaction between APP rG4 wt region and DHX36 protein.

FIG. 4C Inhibition curve obtained from FIG. 4B. The IC₅₀ value was determined to be 13.1±1.2 nM.

FIG. 4D DHX36 overexpression up-regulated APP expression, while L-Apt.4-1c-ASO15nt_{(APP)} rescued the effect of DHX36 detected by western blotting. GAPDH is used as loading control.

FIG. 4E Normalized APP expression results obtained from FIG. 4D. Results were obtained from three replicates. Error bars represented the standard error of mean. *, P<0.05.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

As used herein, the term “L-form RNA” (L-RNA) refers to an artificial RNA built from L-ribose. Compared to naturally occurring oligonucleotides (e.g., D-form RNA, D-RNA), which are homochiral and are built from D-ribose, L-RNA is an enantiomeric counterpart of the natural oligonucleotide and is artificially synthesized via chemical reactions by using L-ribose as the major stating material.

As used herein, the term “aptamer” refers to an oligonucleotide (e.g., a DNA or RNA oligonucleotide) having specific binding regions capable of forming complexes with an intended target molecule in an environment wherein other substances in the same environment are not complexed to the oligonucleotide.

The term “G-quadruplex structure (G4 structure)” as used herein refers to a four-stranded helical nucleic acid structure comprising multiple stacked G-tetrads, each of which consists of four guanine bases that associate in a cyclical manner through Hoogsteen hydrogen bonds and are further stabilized, through coordination to a cation in the center. The body of stacked G-tetrads, comprising a total of 2-8 layers, is collectively referred to as the G-tetrad core. Each of the four guanine columns constituting the G-tetrad core can arise from a single (continuous column), two, or four (discontinuous column) separate guanine stretch/stretches. The term “parallel G-quadruplex”, as used herein, relates to a G-quadruplex (G4) structure wherein all four strands point in the same direction.

The term “antisense oligonucleotide (ASO)” as used herein refers to single stranded DNA or RNA that are complementary to a pre-mRNA or mRNA sequence and can reduce the RNA level thereby reducing the protein level. According to preferred embodiments of the present disclosure, the present ASO is single stranded DNA reverse complementary to the 3′-flanking sequence of the APP untranslated region (UTR) rG4 structure.

### II. The Present L-RNA Aptamer-ASO Conjugate

The present disclosure therefore is directed to an L-form ribonucleic acid (L-RNA) aptamer-antisense oligonucleotide (ASO) conjugate having the structure of formula (I),

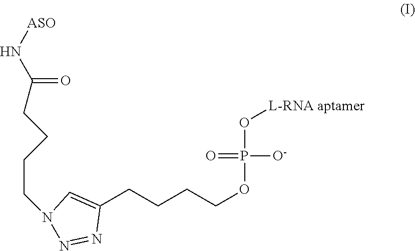

wherein,
the L-RNA aptamer comprises a ribonucleic acid sequence of SEQ ID NO: 1; and
the ASO comprises a deoxyribonucleic acid sequence selected from the group consisting of SEQ ID NOs: 2, 3 or 4.

According to embodiments of the present disclosure, the L-RNA aptamer is about 25 nucleotides in length and comprises a ribonucleic acid 100% identical to SEQ ID NO: 1.

According to embodiments of the present disclosure, the ASO is about 10 to 20nucleotides in length and comprises a deoxyribonucleic acid that is any one of SEQ ID NOs: 2, 3 or 4. In some embodiments, the ASO has 10 nucleotides in length and is the deoxyribonucleic acid sequence of SEQ ID NO: 2. In further embodiments, the ASO has 15 nucleotides in length and is the deoxyribonucleic acid sequence of SEQ ID NO: 3. In other embodiments, the ASO has 20 nucleotides in length and is the deoxyribonucleic acid sequence of SEQ ID NO: 4.

According to embodiments of the present disclosure, the L-RNA aptamer-ASO conjugate could recognize an amyloid precursor protein (APP) 3′-untranslated region (UTR) RNA G-quadruplexes (rG4) structure. Thus, the present L-RNA aptamer-ASO conjugate could serve as a tool for identifying rG4 structure in APP gene.

Optionally or in addition, the L-RNA aptamer-ASO conjugate further includes a fluorescein molecular disposed at the 5′-end of the ASO thereby conferring the L-RNA aptamer-ASO conjugate to be viewed under a fluorescent microscope.

The present L-RNA aptamer-ASO conjugate may be synthesized by any known method, or by procedures described in working examples of the present disclosure.

### III. Uses of the Present L-RNA Aptamer-ASO Conjugate

### (i) In Vitro Imaging

As the present L-RNA aptamer-ASO conjugate could specifically recognize APP rG4 structure, thus it may serve as a tool for identifying rG4 structure in the APP gene. Accordingly, another aspect of the present disclosure aims at providing a method of imaging APP rG4 in a cell. The method includes steps of,
(a) transfecting the cell with a messenger RNA of APP;
(b) permeating the cell,
(c) contacting the permeated cell of step (b) with a cyanine3 (Cy3) labeled APP nucleic acid probe and the present L-RNA aptamer-ASO conjugate, which comprises a fluorescein molecule disposed at the 5′-end of the ASO; and
(d) subjecting the product of step (c) to fluorescence microscopy analysis to produce an image of the APP rG4 in the cell.

According to embodiments of the present disclosure, the cell is first transfected with the APP mRNA; then the cell is permeated by a non-ionic surfactant, such as polyethylene glycol octyl phenyl ether (which is generally known as “TX-100”) (step (b)). Once the cell is permeated, then, an APP nucleic acid probe having been labeled with Cy3 and the present L-RNA aptamer-ASO conjugate having been labeled with fluorescein are incubated with the permeated cell for a sufficient period of time (step (c)), to allow both the APP nucleic acid probe and the present L-RNA aptamer-ASO conjugate to enter the cell and bind to the rG4 structure of the APP gene, thereby allowing the rG4 structure to be visualized under a fluorescence microscope (step (d)).

### (ii) In Vitro Suppressing Endogenous APP Expression

According to certain embodiments of the present disclosure, the present L-RNA aptamer-ASO conjugate is found to suppress APP protein expression. Thus, the present L-RNA aptamer-ASO conjugate may serve as a tool for regulating the APP gene expression.

A further aspect of the present disclosure thus aims at providing a method of suppressing the expression of APP in a cell. The method includes transfecting the cell with a sufficient amount of the present L-RNA aptamer-ASO conjugate to reduce the endogenous level of APP in the cell. According to embodiments of the present disclosure, the transfection is achieved by using lipofectamine. According to further embodiments of the present disclosure, the present L-RNA aptamer-ASO conjugate suppresses the APP protein expression in dose-dependent and time-dependent manners, in which the strongest inhibition of APP expression occurred after treatment for 22 hrs.

(iii) Treatment Method

Since the proteolysis of APP will generate amyloid beta (Aβ), a polypeptide whose amyloid fibrillar form is the primary component of amyloid plaques found in the brains of Alzheimer's disease patients. Accordingly, the present L-RNA aptamer-ASO conjugate may also be used as an agent for treating neurodegenerative disease, as it could suppress the production of endogenous APP.

Examples of neurodegenerative disease that may be treated by L-RNA aptamer-ASO conjugate include, but are not limited to, Alzheimer's disease, Fragile X Syndrome (FXS), Amyotrophic Lateral Sclerosis/Frontal-temporal Dementia (ALS/FTD), Parkinson's disease (PD), and the like.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

L-Apt.4-1c_5′Hexynyl was synthesized by Chemgenes. Antisense oligonucleotide (ASO) deoxyribonucleic acid (DNA)_3′Azide and ASO DNA_5′FAM-3′Azide were bought from Integrated DNA Technologies (IDT). RHAU53 peptide was synthesized by Synpeptide. DHX36 protein was bought from OriGene. pcDNA3.1-3×Flag-C was bought from Youbio and DHX36 ORF was synthesized and inserted into pcDNA3.1-3×Flag-C to generate DHX36-pcDNA3.1-3×Flag-C by Youbio.

### Click Reaction

Alkyne-modified aptamer (L-Apt.4-1c_5′Hexynl, SEQ ID NO: 1) and azide-modified oligos (ASO 10nt_DNA_3′Azide, ASO 15nt_DNA_3′Azide and ASO 20nt_DNA_3′Azide; SEQ ID NOs: 2, 3, and 4), triethylammonium acetate (TEAA), freshly prepared ascorbic acid, Cu (II)-[tris (benzyl triazolyl methyl) amine] (TBTA), dimethyl sulfoxide (DMSO) were mixed and degassed by nitrogen gas. Then, click reaction was performed in a thermoshaker at 40° C. for 4-6 h with shaking at 1300 rpm. After that, the products were resolved on 15% denaturing gel at 300V for 40 min in 1×Tris-borate-ethylenediaminetetraacetic acid (TBE) buffer. Then, the product bands were cut, and the gel was crushed and suspended in homemade TEL800 buffer (1×Tris-ethylenediaminetetraacetic acid, pH 8.0, 0.8M LiCl) with shaking at 1300 rpm overnight at 4° C. Then, conjugate was recovered by ribonucleic acid (RNA) clean and concentrator-5 (Zymo research) following manufacturer instructions. To assess the purity of the conjugates, products were resolved in 15% denaturing gel, stained with SYBR Gold and scanned by ChemiDoc Touch Imaging System (Bio-Rad).

| | Final | |
|---|---|---|
| Reagent | concentration | |
| L-Apt.4-1c_5′Hexynyl | 80 | μM |
| ASO DNA_3′ Azide | 320 | μM |
| DMSO | 45 | vol % |
| Ascorbic acid | 0.5 | mM |
| Cu (II)-TBTA | 0.5 | mM |

| | |
|---|---|
| TEAA | 0.2M |

### Matrix-Assisted Laser Desorption Ionization-Time of Flight Mass Spectrometry (MALDI-TOF MS)

To make the matrix, ammonium citrate dibasic and 2′,6′-Dihydroxyacetophenone were dissolved in 50% methanol to saturation. Then, conjugate oligos and matrix were mixed with the ratio of 1:1. After that, samples were loaded on the MALDI plate and dried in the fume hood. Then, the plate was loaded to the 4800 Plus MALDI TOF/TOF Analyzer (Applied Biosystems) for detection.

### Electrophoretic Mobility Shift Assay (EMSA)

For structure refolding, FAM/HEX RNA oligos, L-aptamer or L-Apt.4-1c-ASO conjugates were heated at 95° C. for 5 min, 25° C. for 5 min, then 4° C. for at least 30 min separately in buffer containing 150 mM KCl, 1 mM MgCl₂, 25 mM Tris-HCl (pH 7.5) and 8% sucrose. If dissociation constant (K_{d}) is calculated, L-aptamer or conjugates were serial diluted before heating. After refolding, RNA oligos and conjugates/L-aptamer were mixed and incubated for 30 min at 37° C. for binding. Then, the samples were loaded and resolved in 6% (19:1, acrylamide/bis-acrylamide) native gel for 70 min at 35 mA at 4° C. in the running buffer containing 50 mM KOAc, 25 mM Tris-HCl (pH 7.5), 1 mM MgCl₂. At last, gels were scanned using Typhoon laser-scanner platform (Cytiva). Gel pictures were analyzed by image J software.

### EMSA for Competition Assay

L-Apt.4-1c-ASO15nt_{(APP)} conjugate was serial diluted, and then the conjugate and HEX APP rG4 wt region were firstly refolded by heating at 95° C. for 5 min, 25° C. for 5 min, then 4° C. for at least 30 min separately in buffer containing 150 mM KCl, 1 mM MgCl₂, 25 mM Tris-HCl (pH 7.5) and 8% sucrose separately. 48 nM DHX36 protein (SEQ ID NO: 41) (or 100 nM RHAU53 peptide (SEQ ID NO: 42)), HEX_APP rG4 wt region (SEQ ID NO: 12) and L-Apt.4-1c-ASO15nt_{(APP)} were mixed and incubated for 30 min at 37° C. for competition binding. Then, the samples were loaded and resolved in 6% (37.5:1, acrylamide/bis-acrylamide) native gel for 70min at 35 mA at 4° C. in the running buffer containing 50 mM KOAc, 25 mM Tris-HCl (pH 7.5), 1 mM MgCl₂ (for DHX36) or running at 12.5 mA for 75 min in buffer containing 0.5×TBE and 40 mM KOAc (for RHAU53). At last, gels were scanned using Typhoon laser-scanner platform (Cytiva). Gel pictures were analyzed by image J software.

### EMSA for Unwinding Assay

For structure refolding, FAM_APP rG4 region (SEQ ID NO: 9) were subjected to a thermal denaturation step at 95° C. for 5 min, followed by a slow cooling process to reach a temperature of 21° C. at a ramp rate of 0.1° C./s in a buffer solution composed of 150 mM KCl, 2 mM MgCl₂ and 25 mM Tris-HCl (pH 7.5). For marker preparation, 100 nM of single-stranded RNA (ssRNA) trap (SEQ ID NO: 27) was added before refolding, and 150 mM LiCl instead of KCl in buffer was used to facilitate annealing. 0.11 μg of DHX36 protein was introduced into the system for 10 min for binding at 37° C. Then, 500 nM of ssRNA trap, and 100 nM of adenosine triphosphate (ATP) or adenylyl-imidodiphosphate (AMP-PNP) (non-hydrolysable analog of ATP) were introduced to incubate for 10 min at 37° C. for unwinding. Then, 10 μg of proteinase K was added and incubated at 37° C. for 40 min to digest DHX36 protein. Afterwards, samples were loaded and resolved in 13% (19.5:1, acrylamide/bis-acrylamide) native gel for 80 min at 35 mA at 4° C. in the running buffer containing 0.5×TBE and 40 mM KOAc. At last, gels were scanned using Typhoon laser-scanner platform (Cytiva).

### Microscale Thermophoresis (MST)

L-Apt.4-1c_5′Hexynyl or L-Apt.4-1c-ASO conjugates were serial diluted to 16 sets. Then L-Apt.4-1c_5′Hexynyl or conjugates and FAM_RNA oligos were subjected to a thermal denaturation by heating to 95° C. for 5 min, and then cooled to 25° C. for 5 min, and finally chilled 4° C. for at least 30 min in the buffer solution (25 mM Tris-HCl (pH 7.5), 150 mM KCl, 1 mM MgCl₂), separately. After that, FAM_RNA oligos were mixed with the diluted L-Apt.4-1c_5′Hexynyl or conjugates and incubated at 37° C. for 30 min. Samples were subsequently transferred into the Nano-Temper Monolith NT.115 capillary tubes and measured using Monolith NT.115 instrument in blue light mode. Finally, the data was analyzed using the NanoTemper analysis (nta) software to determine the K_{d} value.

### Western Blotting

5-10×10⁴/well Hela cells were cultured and allowed to adhere onto a 24-well plate. Following a 24 h incubation period, transfection was performed to deliver the oligos (L-Apt.4-1c-ASO_{(APP)} conjugates, L-Apt.4-1c_5′Hexynyl, ASO DNA) into the cells by lipofectamine 2000. After transfection for indicated time, cells were collected and disrupted using a lysis buffer containing 50 mM Tris-HCl (pH 7.5), 1% Triton X-100, 250 mM NaCl, and 5 mM ethylenediaminetetraacetic acid (EDTA). After that, sample lysates were boiled with sample buffer (Bio-Rad) and resolved by 8% Sodium dodecyl sulfate-polyacrylamide gel electrophoresis (SDS-PAGE). Western blotting was performed as published before1. Briefly, the key steps involved gel running, transfer of proteins to membrane, blocking to avoid non-specific binding, primary antibody incubation, washing away unbound antibodies, secondary antibody incubation, further washing, ECL substrate for signal generation. At last, the membranes were scanned by ChemiDoc Touch Imaging System (Bio-Rad). The protein intensity was analyzed utilizing Image Lab software (Bio-Rad). APP antibody (Millipore Sigma, MAB348) was diluted in 10% milk (1:1000) and GAPDH antibody (Santa Cruz Biotechnology, sc-32233) in 5% milk (1:1000).

### Cell Imaging

RNA containing the rG4 sequence were produced through in vitro transcription by T7High Yield RNA Synthesis Kit (NEB) following manufacturer's protocol. Briefly, imaging-forward DNA strand (SEQ ID No: 28) and imaging-reverse DNA strand (SEQ ID NO: 29, 30, 31 or 32) were hybridized by incubating at 95° C. for 5 min, and 4° C. for several hours to form the DNA template for transcription. Then transcription was conducted at 37° C. for 3.5 h, and DNA template was digested by Turbo DNase. After resolving by 12% denaturing gel, the RNAs were crushed and suspended in homemade TEL800 buffer with shaking at 1300 rpm overnight at 4° C. Next day, RNAs were recovered from the gel using the RNA clean and concentrator-5 kit provided by Zymo Research following manufacturer instructions. Hela cells were cultured and allowed to adhere onto 35 mm confocal dishes. Following the 24 h incubation period, the Hela cells were transfected with the in vitro transcribed RNAs by lipofectamine 2000. After 7 h transfection, the cells underwent fixation first, through treatment with 4% paraformaldehyde for 15 min, and subsequently, the cell membranes were permeabilized by exposure to 0.5% Triton X-100 for 30 min, washed by 2×saline-sodium citrate (SSC) and stained with Cy3-probe and FAM_L-Apt.4-1c-ASO15nt_{(APP)} sequentially, which were diluted in buffer containing 4×SSC, 30% deionized-formamide, 10% dextran sulfate, 0.5 mM EDTA. After 2×SSC washing for 5 times, the cells were exposed to a 5 μg/ml solution of the Hoechst 33342 dye for 15 min incubation period and scanned by confocal microscopy (Leica).

For RNase H1 subcellular distribution detection. Hela cells were cultured and allowed to adhere onto 35 mm confocal dishes for 24 h. Then The cells underwent fixation first, through treatment with 4% paraformaldehyde for 15 min, and subsequently, the cell membranes were exposed to 0.3% Triton X-100 for 10 min for permeabilization. Then cells were washed with phosphate buffered saline (PBS) and blocked with 1% bovine serum albumin (BSA) for 30 min. RNase H1 primary antibody (Proteintech 15606-1-AP, 1:100 in 1% BSA) was incubated with cells at 4° C. After overnight incubation, cells were subjected to 5 rounds of PBS washing and treated with Alexa Fluor® 555 secondary antibody (Abcam ab150078, 1:500 in 1% BSA at room temperature for 1 h. Then after PBS washing for 5 times, and Hoechst 33342 (5 μg/ml) staining for 20 min, cells were scanned by confocal microscopy (Leica).

### RNase H Cleavage Assay

HEX_APP rG4 wt region and L-Apt.4-1c-ASO15nt_{(APP)} were firstly refolded by heating at 95° C. for 5 min, 25° C. for 5 min, then 4° C. for at least 5 min independently in the buffer formulated with 25 mM Tris-HCl (pH 7.5), 150 mM KCl, 1 mM MgCl₂. A mixture of HEX APP rG4 wt region and L-Apt.4-1c-ASO15nt_{(APP)} was prepared and incubated at 37° C. for 10 min in a buffer solution comprising 50 mM Tris-HC1, 75 mM KCl, 3 mM MgCl₂ and 10 mM DTT, representing the 1×RNase H Reaction Buffer. After that, indicated amount of RNase H was added to react at 37° C. At indicated time, 25 mM EDTA and equal volume of formamide was added to stop the reaction. At last, prior to separation, the samples were denatured by incubating them at 95° C. for 3 min, and subsequently resolved on a 15% urea-containing denaturing gel. Finally, gels were scanned using Typhoon laser-scanner platform (Cytiva).

### Reverse Transcription-Quantitative Polymerase Chain Reaction (RT-qPCR)

After harvesting the Hela cells, RNAs were isolated employing MiniBEST Universal RNA Extraction Kit (TaKaRa), in accordance with the step-by-step instructions provided by the manufacturer. Then total RNAs (100-150 ng) were reverse transcribed into cDNA utilizing

PrimeScript RT reagent kit (Perfect Real Time) (Takara RR037Q), and subsequently subjected to qPCR analysis utilizing SsoAdvanced Universal SYBR Green Supermix (Bio-Rad) with APP or GAPDH primers (SEQ ID NOs: 36-39). Finally, samples were processed and analyzed using CFX connect real-time system (Bio-Rad).

The oligonucleotide sequences used in the present disclosure are provided in Table 1; and the protein and peptide sequences are provided in Table 2.

TABLE 1
Oligonucleotide sequences

| Name | Sequences (5′-3′) | SEQ ID NO |
|---|---|---|
| L-Apt.4- | L-Hexynyl-GCCCUAAAGGUGGUGGUGGGAGGGC | 1 |
| lc_5′Hexynyl | | |
| ASO | CAGCAGAGCA-Azide | 2 |
| 10 nt_DNA_3′Azide | | |
| ASO | AAGACCAGCAGAGCA-Azide | 3 |
| 15 nt_DNA_3′Azide | | |
| ASO 20 nt_ | AATTGAAGACCAGCAGAGCA-Azide | 4 |
| DNA_3′Azide | | |
| ASO 15 nt_DNA- | FAM-AAGACCAGCAGAGCA-Azide | 5 |
| 5′FAM-3′Azide | | |
| FAM_TRF2 rG4 | FAM- | 6 |
| region | GGGAGGGCGGGGAGGGCGCGCGGCGAUCGGACACGA | |
| FAM_MT3-MMP rG4 | FAM- | 7 |
| region | GAGGGAGGGAGGGAGAGGGAGAGAGGGAGAAAACG | |
| | AAGGG | |
| FAM_Bcl2 rG4 region | FAM- | 8 |
| | GGGGGCCGUGGGGUGGGAGCUGGGGCGAGAGGUGCC | |
| | GUUGGCCCC | |
| FAM_APP rG4 wt | FAM- | 9 |
| region | GGGGCGGGUGGGGAGGGGUGCUCUGCUGGUCUUCAA | |
| | UU | |
| FAM_APP rG4 wt | FAM-CGGGGCGGGUGGGGAGGGGU | 10 |
| motif | | |
| FAM_APP rG4 mut | FAM-CGAAGCGAGUGAAGAGAAGU | 11 |
| motif | | |
| HEX_APP rG4 wt | HEX- | 12 |
| region | GGGGCGGGUGGGGAGGGGUGCUCUGCUGGUCUUCAA | |
| | UU | |
| HEX_APP rG4 mut | HEX- | 13 |
| region | GAAGCGAGUGAAGAGAAGUGCUCUGCUGGUCUUCAA | |
| | UU | |
| FAM_c-Kit 1 dG4 | FAM- | 14 |
| region | GGAGGGCGCTGGGAGGAGGGGCTGCTGCTCGCCGCTC | |
| | GCG | |
| FAM_hTERC dG4 | FAM- | 15 |
| region | GGGTTGCGGAGGGTGGGCCTGGGAGGGGTGGTGGCC | |
| | A | |
| FAM_hTELO dG4 | FAM- | 16 |
| region | TTAGGGTTAGGGTTAGGGTTAGGGTTAGAGTTAGAGTT | |
| | AGAGTT | |
| FAM_Bcl2 Mid dG4 | FAM- | 17 |
| region | GGGCGCGGGAGGAAGGGGGGGGAGCGGGGCTGTGG | |
| | TGCCTGT | |
| FAM_VEGF dG4 | FAM- | 18 |
| region | GGGGCGGGCCGGGGGCGGGGTCCCGGCGGGGCGGAG | |
| | CCAT | |
| FAM_Bcl2 dG4 | FAM- | 19 |
| region | GGGGGCCGTGGGGTGGGAGCTGGGGCGAGAGGTGCC | |
| | GTTGGCCCC | |
| FAM_MT3-MMP dG4 | FAM- | 20 |
| region | GAGGGAGGGAGGGAGAGGGAGAGAGGGAGAAAACG | |
| | AAGGG | |
| FAM_TRF2 dG4 | FAM- | 21 |
| region | GGGAGGGCGGGGAGGGCGCGCGGCGATCGGACACGA | |
| poly rA | FAM-AAAAAAAAAAAAAAAAAA | 22 |
| poly rC | FAM-CCCCCCCCCCCCCCCCCC | 23 |
| poly rU | FAM-UUUUUUUUUUUUUUUUUU | 24 |
| DNA hairpin | FAM-CAGTACAGATCTGTACTG | 25 |
| RNA hairpin | FAM-CAGUACAGAUCUGUACUG | 26 |
| ssRNA trap | UCCCCACCCGCCCCG | 27 |
| T7-imaging-forward | TAATACGACTCACTATAG | 28 |
| DNA strand | | |
| APP wt-imaging- | TACAATCATCCTGCAGAAAATTGTTTTGGAGAATTCTT | 29 |
| reverse DNA strand | GGTAATTGAAGACCAGCAGAGCACCCCTCCCCACCCG | |
| | CCCCGTAAAAGTGCTTACAATGAACAGGGATTCTTTTC | |
| | TTTATCAAAGACTATAGTGAGTCGTATTA | |
| APP rG4 region | TACAATCATCCTGCAGAAAATTGTTTTGGAGAATTCTT | 30 |
| deleted-imaging- | GGTAATTGGTAAAAGTGCTTACAATGAACAGGGATTCT | |
| reverse DNA strand | TTTCTTTATCAAAGACTATAGTGAGTCGTATTA | |
| TRF2-imaging- | TGCCCGCCCGCTGCCGTCGCTACTCCCGCCTCCTCCCG | 31 |
| reverse DNA strand | CCATCGTGTCCGATCGCCGCGCGCCCTCCCCGCCCTCC | |
| | CGGCCGGGCCGCTTCCTCGGCTGTGACGCCGCTGGGT | |
| | CACGCACGACTATAGTGAGTCGTATTA | |
| MT3-MMP-imaging- | TAAGATCATAGTGAACTGTGCTTCAATGGATGGACGAG | 32 |
| reverse DNA strand | CTCCCCTTCGTTTTCTCCCTCTCTCCCTCTCCCTCCCTC | |
| | CCTCGTTTCCTTTCAAAAAAAAGTCCTCCGGGTGGGTA | |
| | AGGAGCCTGCTATAGTGAGTCGTATTA | |
| APP Cy3-probe | Cy3-TACAATCATCCTGCAGAAAATTGTT | 33 |
| TRF2 Cy3-probe | Cy3-TGCCCGCCCGCTGCCGTCGCTACTC | 34 |
| MT3-MMP Cy3-probe | Cy3-TAAGATCATAGTGAACTGTGCTTCA | 35 |
| GAPDH-F | GGAGCGAGATCCCTCCAAAAT | 36 |
| GAPDH-R | GGCTGTTGTCATACTTCTCATGG | 37 |
| APP-F | GTGCTCTGCTGGTCTTCAAT | 38 |
| APP-R | GAAAAGTCTTGCCCGGGGTT | 39 |
| DHX36 ORF | ATGAGTTATGACTACCATCAGAACTGGGGCCGTGATGG | 40 |
| | GGGTCCCCGCAGCTCCGGTGGGGGCTATGGAGGGGGG | |
| | CCAGCAGGGGGTCATGGAGGTAACCGAGGCTCCGGAG | |
| | GAGGCGGCGGCGGCGGAGGGGGTGGTCGAGGCGGCA | |
| | GGGGCCGGCATCCCGGGCACCTGAAAGGCCGCGAAAT | |
| | CGGCATGTGGTACGCGAAAAAACAGGGGCAGAAGAA | |
| | CAAGGAAGCGGAGAGGCAAGAGAGAGCTGTAGTACA | |
| | CATGGATGAACGACGAGAAGAACAAATTGTACAGTTA | |
| | CTGAATTCTGTTCAAGCGAAGAATGATAAAGAGTCAG | |
| | AAGCACAGATATCCTGGTTTGCTCCTGAGGATCATGGA | |
| | TACGGTACTGAAGTTTCTACTAAGAACACACCATGCTC | |
| | AGAGAACAAACTTGACATCCAGGAAAAGAAGTTGATA | |
| | AATCAAGAAAAAAAAATGTTTAGAATCAGGAACAGAT | |
| | CATATATTGACCGAGATTCTGAGTATCTCTTGCAAGAAA | |
| | ATGAACCAGATGGAACTTTAGACCAAAAATTATTGGAA | |
| | GATTTACAAAAGAAAAAAAATGACCTTCGGTATATTGA | |
| | AATGCAGCATTTCAGAGAAAAGCTGCCTTCGTATGGAA | |
| | TGCAAAAGGAATTGGTAAATTTAATTGATAACCATCAG | |
| | GTAACAGTAATAAGTGGTGAAACTGGTTGTGGCAAAA | |
| | CCACTCAAGTTACTCAGTTCATTTTGGATAACTACATTG | |
| | AAAGAGGAAAAGGATCTGCTTGCAGAATAGTTTGTAC | |
| | TCAGCCAAGAAGAATTAGTGCCATTTCAGTTGCGGAA | |
| | AGAGTAGCTGCAGAAAGGGCAGAATCTTGTGGCAGTG | |
| | GTAATAGTACTGGATATCAAATTCGTCTCCAGAGTCGGT | |
| | TGCCAAGGAAACAGGGTTCTATCTTATACTGTACAACA | |
| | GGAATCATCCTTCAGTGGCTCCAGTCAGACCCGTATTT | |
| | GTCCAGTGTTAGTCATATCGTACTTGATGAAATCCATGA | |
| | AAGAAATCTGCAGTCAGATGTTTTAATGACTGTTGTTA | |
| | AAGACCTTCTCAATTTTCGATCTGACTTGAAAGTAATAT | |
| | TGATGAGTGCAACATTGAATGCAGAAAAGTTTTCAGA | |
| | ATATTTTGGTAACTGTCCAATGATACATATACCTGGTTTT | |
| | ACCTTTCCGGTTGTGGAATATCTTTTGGAAGATGTAATT | |
| | GAAAAAATAAGGTATGTTCCAGAACAAAAAGAACACA | |
| | GATCCCAGTTTAAGAGGGGTTTCATGCAAGGGCATGTA | |
| | AATAGACAAGAAAAAGAAGAAAAAGAAGCAATATATA | |
| | AAGAACGTTGGCCAGATTATGTAAGGGAACTGCGAAG | |
| | AAGGTATTCTGCAAGTACTGTAGATGTTATAGAAATGAT | |
| | GGAGGATGATAAAGTTGATCTGAATTTGATTGTTGCCC | |
| | TCATCCGATACATTGTTTTGGAAGAAGAGGATGGTGCG | |
| | ATACTGGTCTTTCTGCCAGGCTGGGACAATATCAGCAC | |
| | TTTACATGATCTCTTGATGTCACAAGTAATGTTTAAATC | |
| | AGTTAACCAGACACAGGTGTTTAAAAGAACCCCTCCT | |
| | GGTGTTCGGAAAATAGTAATTGCTACCAACATTGCGGA | |
| | GACTAGCATTACCATAGATGATGTCGTTTATGTGATAGA | |
| | TGGAGGAAAAATAAAAGAGACACATTTTGATACTCAG | |
| | AACAATATCAGTACAATGTCCGCTGAGTGGGTTAGTAA | |
| | AGCTAATGCCAAACAGAGAAAAGGTCGAGCTGGAAG | |
| | AGTTCAACCTGGTCATTGCTATCATCTGTATAATGGTCT | |
| | TAGAGCAAGTCTTCTAGATGACTATCAACTGCCAGAAA | |
| | TTTTGAGAACTCCTTTGGAAGAACTTTGTTTACAAATA | |
| | AAGATTTTAAGGCTAGGTGGAATTGCTTATTTTCTGAGT | |
| | AGATTAATGGACCCACCATCAAATGAGGCAGTGTTACT | |
| | CTCCATAAGACACCTGATGGAGCTGAACGCTTTGGATA | |
| | AACAAGAAGAATTGACACCTCTTGGAGTCCACTTGGC | |
| | ACGATTACCCGTTGAGCCACATATTGGAAAAATGATTC | |
| | TTTTTGGAGCACTGTTCTGCTGCTTAGACCCAGTACTC | |
| | ACTATTGCTGCTAGTCTCAGTTTCAAAGATCCATTTGTC | |
| | ATTCCACTGGGAAAAGAAAAGATTGCAGATGCAAGAA | |
| | GAAAGGAATTGGCAAAGGATACTAGAAGTGATCACTT | |
| | AACAGTTGTGAATGCGTTTGAGGGCTGGGAAGAGGCT | |
| | AGGCGACGTGGTTTCAGATACGAAAAGGACTATTGCT | |
| | GGGAATATTTTCTGTCTTCAAACACACTGCAGATGCTG | |
| | CATAACATGAAAGGACAGTTTGCTGAGCATCTTCTTGG | |
| | AGCTGGATTTGTAAGCAGTAGAAATCCTAAAGATCCAG | |
| | AATCTAATATAAATTCAGATAATGAGAAGATAATTAAAG | |
| | CTGTCATCTGTGCTGGTTTATATCCCAAAGTTGCTAAAA | |
| | TTCGACTAAATTTGGGTAAAAAAAGAAAAATGGTAAA | |
| | AGTTTACACAAAAACCGATGGCCTGGTTGCTGTTCATC | |
| | CTAAATCTGTTAATGTGGAGCAAACAGACTTTCACTAC | |
| | AACTGGCTTATCTATCACCTAAAGATGAGAACAAGCAG | |
| | TATATACTTGTATGACTGCACAGAGGTTTCCCCATACTG | |
| | TCTCTTGTTTTTTGGAGGTGACATTTCCATCCAGAAGG | |
| | ATAACGATCAGGAAACTATTGCTGTAGATGAGTGGATT | |
| | GTATTTCAGTCTCCAGCAAGAATTGCCCATCTTGTTAA | |
| | GGAATTAAGAAAGGAACTAGATATTCTTCTGCAAGAGA | |
| | AGATTGAAAGTCCTCATCCTGTAGACTGGAATGACACT | |
| | AAATCCAGAGACTGTGCAGTACTGTCAGCTATTATAGA | |
| | CTTGATCAAAACACAGGAAAAGGCAACTCCCAGGAAC | |
| | TTTCCGCCACGATTCCAGGATGGATATTACAGC | |

Note:
mutations are underlined.

TABLE 2
Protein and peptide sequences

| | | SEQ ID |
|---|---|---|
| Name | Sequence | NO |
| DHX36 | MSYDYHQNWGRDGGPRSSGGGYGGGPAGGHGGNRGSGGGGGG | 41 |
| protein | GGGGRGGRGRHPGHLKGREIGMWYAKKQGQKNKEAERQERAVV | |
| | HMDERREEQIVQLLNSVQAKNDKESEAQISWFAPEDHGYGTEVST | |
| | KNTPCSENKLDIQEKKLINQEKKMFRIRNRSYIDRDSEYLLQENEP | |
| | DGTLDQKLLEDLQKKKNDLRYIEMQHFREKLPSYGMQKELVNLID | |
| | NHQVTVISGETGCGKTTQVTQFILDNYIERGKGSACRIVCTQPRRIS | |
| | AISVAERVAAERAESCGSGNSTGYQIRLQSRLPRKQGSILYCTTGIIL | |
| | QWLQSDPYLSSVSHIVLDEIHERNLQSDVLMTVVKDLLNFRSDLK | |
| | VILMSATLNAEKFSEYFGNCPMIHIPGFTFPVVEYLLEDVIEKIRYVP | |
| | EQKEHRCQFKRGFMQGHVNRQEKEEKEAIYKERWPDYVRELRRR | |
| | YSASTVDVIEMMEDDKVDLNLIVALIRYIVLEEEDGAILVFLPGWD | |
| | NISTLHDLLMSQVMFKSDKFLIIPLHSLMPTVNQTQVFKRTPPGVR | |
| | KIVIATNIAETSITIDDVVYVIDGGKIKETHFDTONNISTMSAEWVS | |
| | KANAKQRKGRAGRVQPGHCYHLYNGLRASLLDDYQLPEILRTPLE | |
| | ELCLQIKILRLGGIAYFLSRLMDPPSNEAVLLSIRHLMELNALDKQE | |
| | ELTPLGVHLARLPVEPHIGKMILFGALFCCLDPVLTIAASLSFKDPF | |
| | VIPLGKEKIADARRKELAKDTRSDHLTVVNAFEGWEEARRRGFRY | |
| | EKDYCWEYFLSSNTLQMLHNMKGQFAEHLLGAGFVSSRNPKDPE | |
| | SNINSDNEKIIKAVICAGLYPKVAKIRLNLGKKRKMVKVYTKTDGL | |
| | VAVHPKSVNVEQTDFHYNWLIYHLKMRTSSIYLYDCTEVSPYCLLF | |
| | FGGDISIQKDNDQETIAVDEWIVFQSPARIAHLVKELRKELDILLQE | |
| | KIESPHPVDWNDTKSRDCAVLSAIIDLIKTQEKATPRNFPPRFQDGY | |
| | YSTRTRPLEQKLISEEDLAANDILDYKDDDDKV | |
| RHAU53 | HPGHLKGREIGMWYAKKQGQKNKEAERQERAVVHMDERREEQI | 42 |
| peptide | VQLLNSVQAK | |

### Example 1: Synthesis and Characterization of the Present L-Aptamer-ASO Conjugate

The present L-aptamer-ASOs were synthesized in accordance with procedures described in “Materials and methods” section. The present L-aptamer-ASO is composed of two modules: (1) an L-aptamer for the recognition of rG4 motif via structural recognition, and (2) an antisense DNA oligo (ASO sequence) for the recognition of flanking sequence next to the rG4motif via base pair hybridization. To this purpose, L-Apt.4-1c targeting APP rG4, and an ASO sequence reverse complementary to the 3′flanking sequence of the APP rG4 structure were covalently connected via a click reaction. To facilitate the click reaction, the 5′-end of L-Apt.4-1c was modified with hexynyl and the 3′-end of the ASO DNA was modified with azide. Then, click reaction was performed in the presence of ascorbic acid and Cu (II)-TBTA, in which L-Apt.4-1c and ASO DNA were synthetically linked to produce the desired L-aptamer-ASO conjugate. Three L-aptamer-ASO conjugates were successfully synthesized, namely L-Apt.4-1c-ASO10nt_{(App)}, L-Apt.4-1c-ASO15nt_{(APP)}, and L-Apt.4-1c-ASO20nt_{(APP)}.

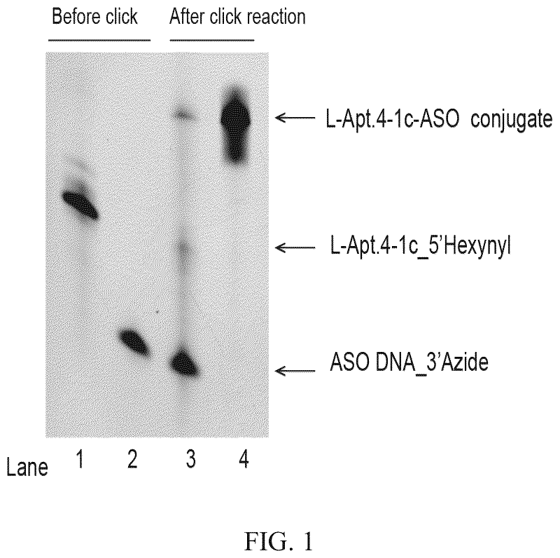

To verify the synthesis, each conjugate thus produced was denatured and then differentiated by electrophoresis. It was found that ASO DNA or conjugates of different sizes showed the correct relative positions in the denatured gel, with larger ones running slower (FIG. 1). Further, MALDI TOF mass spectrometry was used to verify the molecular weight of the conjugates. The results indicated that the calculated mass and the found mass were consistent for all three conjugates. Specifically, the calculated mass and the found mass were respectively 11767.6 and 11767.3 for L-Apt.4-1c-ASO10nt_{(APP)}, 11325.6 and 11329.2 for L-Apt.4-1c-ASO15nt_{(APP)}, and 14905.6 and 14908.4 for L-Apt.4-1c-ASO20nt_{(APP)}, indicating the successful synthesis of the present L-Apt.4-1c-ASO conjugates.

After synthesis of the conjugate, its binding affinity to the APP rG4 wt region (SEQ ID NO: 12) was determined by EMSA. As expected, conjugates with 10nt, 15nt or 20nt ASO (L-Apt.4-1c-ASO10nt_{(APP)}, L-Apt.4-1c-ASO15nt_{(APP)}, and L-Apt.4-1c-ASO20nt_{(APP)}) all significantly improved target binding as compared to that of the L-Apt.4-1c-5′ Hexynyl set, with K_{d} values changed from 60.4±2.6 nM to 11.0±1.2 nM, 0.4±0.1 nM and 0.6±0.1 nM respectively.

The binding between the three L-Apt.4-1c-ASO conjugates and APP rG4 mut region (SEQ ID NO: 13) was also determined. The EMSA results showed that the conjugate binding to the APP rG4 mut region was much weaker than to the APP rG4 wt region (SEQ ID NO: 12), further supporting the importance of the rG4 structure recognition mode. Among the three conjugates, L-Apt.4-1c-ASO15nt_{(APP)} and L-Apt.4-1c-ASO20nt_{(APP)} performed the best. Considering their binding capabilities were similar, L-Apt.4-1c-ASO15nt_{(APP)} was chosen for subsequent experiments.

### Example 2: The Present L-Aptamer—ASO Conjugate Specifically Recognizes a Particular rG4 Region of Interest both In Vitro and in Cells

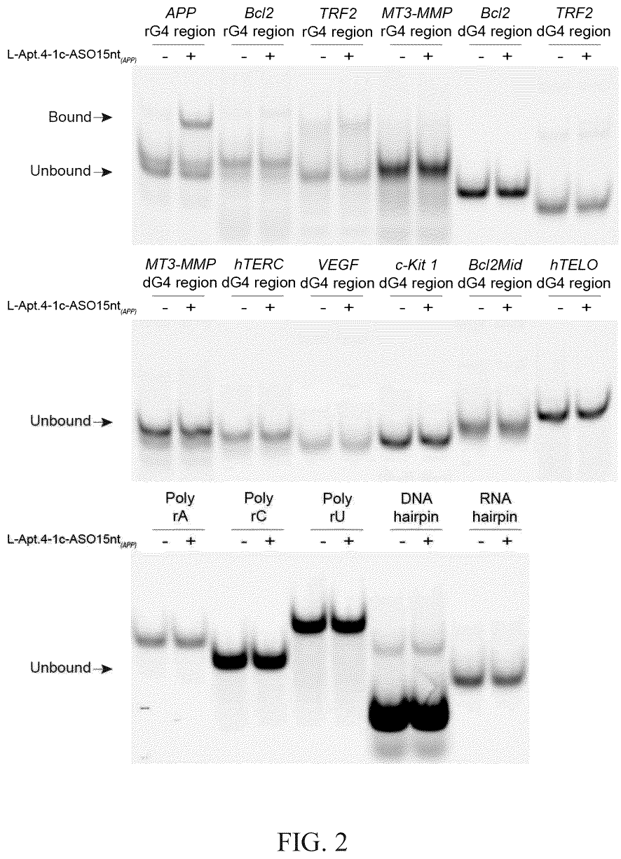

To verify whether the conjugate of Example 1 indeed improved the specificity of target recognition, several rG4s, including Bcl2 rG4 (SEQ ID NO: 8), TRF2 rG4 (SEQ ID NO: 6), and MT3-MMP rG4 (SEQ ID NO: 7), which were previously reported to bind to L-Apt.4-1c, were used as controls for comparison. The EMSA result showed that L-Apt.4-1c-ASO15nt_{(APP)} bound strongly to APP rG4 region, and weakly to Bcl2 rG4, TRF2 rG4, and MT3-MMP rG4 regions, indicating an improvement in binding specificity (FIG. 2).

Furthermore, the binding capacity of Bcl2 rG4 region, TRF2 rG4 region and MT3-MMP rG4 region against L-Apt.4-1c_5′Hexynyl or L-Apt.4-1c-ASO15nt_{(APP)} was compared using EMSA and calculated the K_{d} values. Unlike the significantly enhanced binding to APP rG4 region described above, L-Apt.4-1c-ASO15nt_{(APP)} did not exhibit clear improvement for enhanced binding to other non-target rG4 regions using L-Apt.4-1c_5′Hexynyl as a comparison reference (data not shown). The affinity data obtained with EMSA were further verified by the microscale thermophoresis (MST), which showed a similar result (data not shown). In addition, the binding between L-Apt.4-1c-ASO15nt_{(APP)} and a few DNA G4s (dG4s) regions, including Bcl2 dG4 region (SEQ ID NO: 19), TRF2 dG4 region (SEQ ID NO: 21), MT3-MMP dG4 region (SEQ ID NO: 20), hTERC dG4 region (SEQ ID NO: 15), VEGF dG4 region (SEQ ID NO: 18), c-Kit 1 dG4 region (SEQ ID NO: 14), Bcl2Mid dG4 region (SEQ ID NO: 17), hTELO dG4 region (SEQ ID NO: 16) were also determined, and none of them bound to L-Apt.4-1c-ASO15nt_{(APP)} (FIG. 2).

To investigate the impact of ASO on target binding, the binding of L-Apt.4-1c-ASO15nt_{(APP)} to non-G4 structural targets, including DNA and RNA hairpin (SEQ ID NOs: 25 and 26), polyA/C/U RNAs (SEQ ID NOs: 22-24), and dG4 sequences (SEQ ID NO: 14-21), and none of them showed binding to L-Apt.4-1c-ASO15nt_{(APP)} (FIG. 2), which revealed that the additional ASO sequence introduced in L-Apt.4-1c-ASO did not produce new binding sites for non-target structure recognition. Taken together, the present L-aptamer—D-oligonucleotide conjugate enables the specific recognition of a single rG4 region (APP rG4) in vitro.

To see if the present L-aptamer—D-oligonucleotide conjugate can be applied to distinguish and recognize a particular rG4 structure of interest in cells. We first in vitro transcribed the RNAs containing the rG4 region sequences and then transfected them into Hela cells. In addition, to monitor the intracellular distribution of transfected RNA, Cy3 labeled DNA oligo (Cy3 probe) (SEQ ID NOs: 33-35) complementary to the 3′ end of the RNA was used to hybridize and localize the RNA. To obtain a fluorescently labeled conjugate, FAM-labeled ASO and L-Apt.4-1c_5′Hexynyl were ligated to generate FAM-L-Apt.4-1c-ASO15nt_{(APP)} through a click reaction. After that, fixed and permeabilized Hela cells were stained with FAM-L-Apt.4-1c-ASO15nt_{(APP)} to visualize the APP rG4 region. To demonstrate that FAM-L-Apt.4-1c-ASO15nt_{(APP)} was indeed bound to the APP rG4 region, we also set up APP rG4 and flanking sequence-deleted RNAs (APP rG4 region deleted) (SEQ ID NO: 30) as controls (data not shown). Green FAM fluorescent foci were observed only in the APP wt group, but not in the APP rG4 region-deleted group, and the FAM and Cy3 fluorescent foci were co-localized well, indicating that FAM-L-Apt.4-1c-ASO15nt_{(APP)} correctly recognized the APP rG4 region. In addition, to show the specificity of FAM-L-Apt.4-1c-ASO15nt_{(APP)} in cells, we also tested the non-target rG4 region, including TRF2 (SEQ ID NO: 31) and MT3-MMP (SEQ ID NO: 32) as negative controls. Confocal imaging showed that only the red Cy3 signal of the RNA was detected in the non-target group, while no green FAM-L-Apt.4-1c-ASO15nt_{(APP)} signal was detected at all and no co-localization foci, collectively suggesting specificity recognition of the APP rG4 region (data not shown). Overall, these results confirmed that the FAM-L-Apt.4-1c-ASO conjugate could selectively visualize a specific rG4 region of interest in cells.

### Example 3: L-Apt.4-1c-ASO15nt_{(APP)} Inhibited Endogenous APP Expression in Cells through Translation Inhibition and RNase H-Mediated Knockdown of mRNA

In this example, whether the present conjugate could be used to control endogenous APP gene expression by specifically targeting the APP rG4 region was investigated. To deliver L-Apt.4-1c-ASO15nt_{(APP)} into cells, Lipofectamine 2000 was used for transfection and the APP expression level was determined by western blotting.

First, the effect of different ASO length-conjugates (L-Apt.4-1c-ASO10nt_{(APP)}, L-Apt.4-1c-ASO15nt_{(APP)}, and L-Apt.4-1c-ASO20nt_{(APP)}) on gene expression was tested. The results showed that they all could be transfected into cell and inhibited APP protein expression (data not shown). Among the conjugates, L-Apt.4-1c-ASO15nt_{(APP)} had a stronger inhibitory effect than L-Apt.4-1c-ASO10nt_{(APP)}, while L-Apt.4-1c-ASO15nt_{(APP)} and L-Apt.4-1c-ASO20nt_{(APP)} showed similar inhibitory effects (data not shown), which again supported the use of the 15nt_conjugate for subsequent experiments.

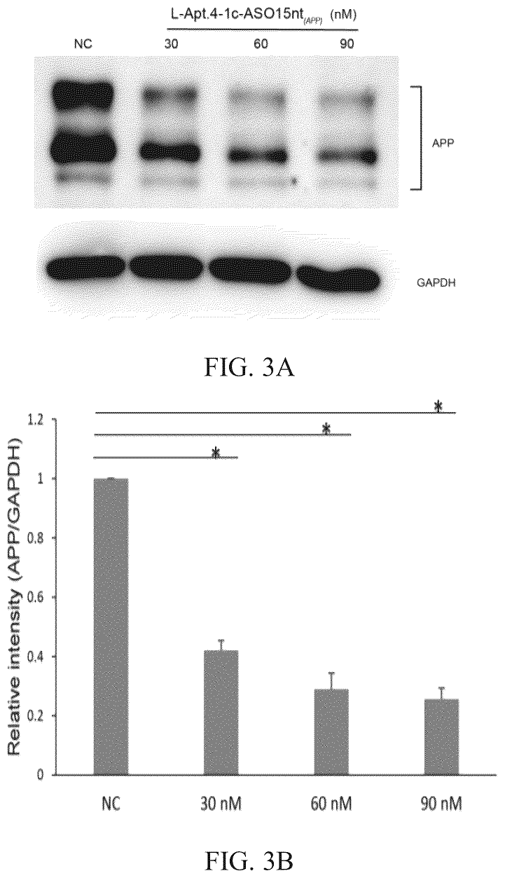

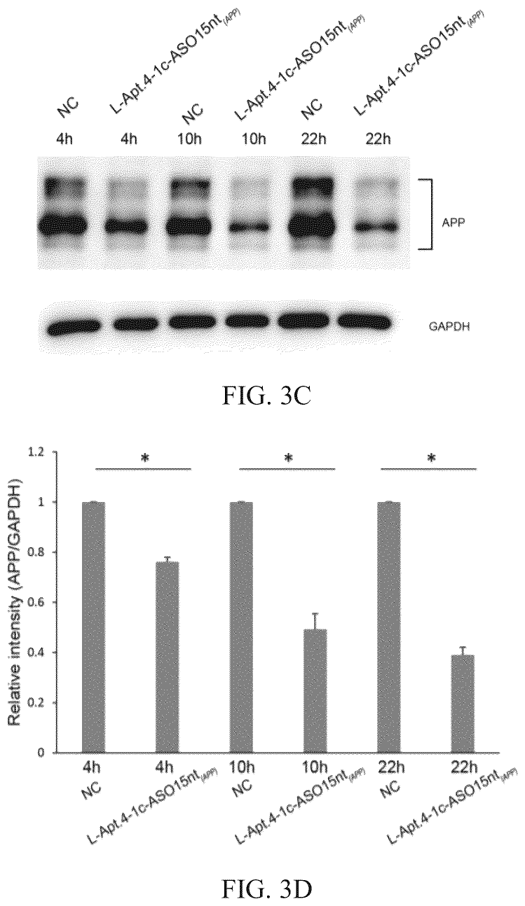

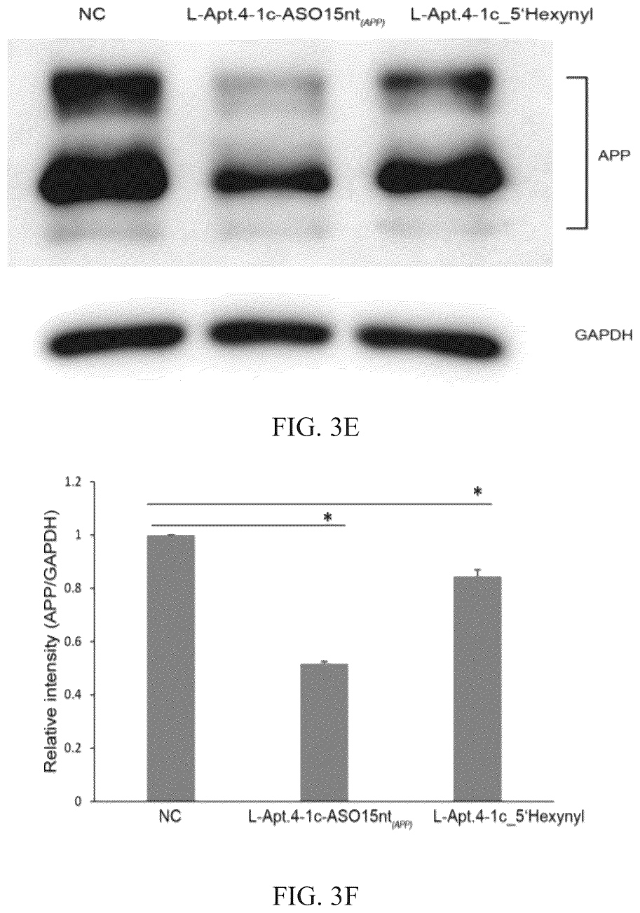

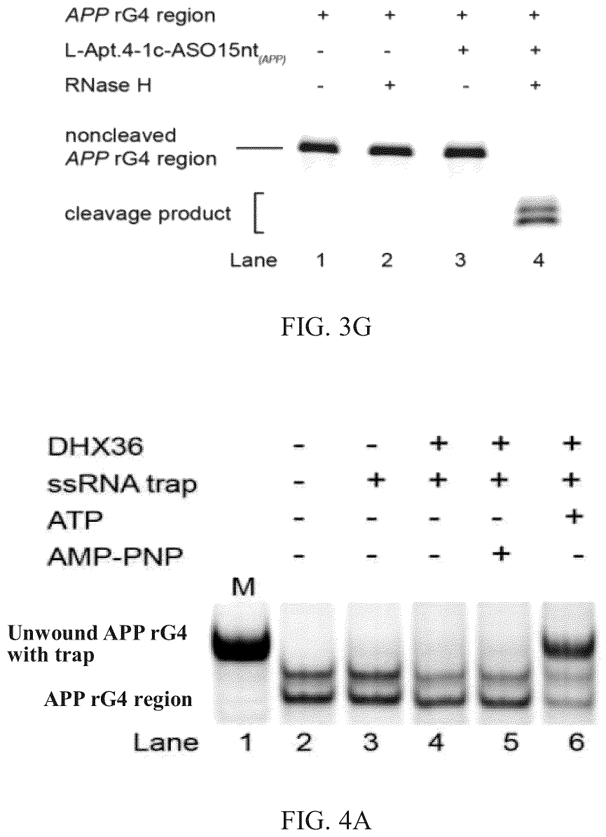

After confirming the use of L-Apt.4-1c-ASO15nt_{(APP)}, cells were then treated with different concentration of L-Apt.4-1c-ASO15nt_{(APP)} (0 nM (NC), 30 nM, 60 nM and 90 nM), and detected the APP protein level. As shown in FIG. 3A and 3B, with increasing concentration of L-Apt.4-1c-ASO15nt_{(APP)}, the APP protein levels decreased accordingly, indicating the inhibition was in a concentration-dependent manner. Different time-point treatments of L-Apt.4-1c-ASO15nt_{(APP)} for 4 h, 10 h and 22 h were also investigated. Compared with NC group (lipofectamine 2000 treated), the inhibition of L-Apt.4-1c-ASO15nt_{(APP)} was already significant for treatment for 4 h. As treatment time increases, the inhibition effect continuously increased until the strongest inhibition effect was reached at 22 h (FIG. 3C and 3D), suggesting a time-dependent inhibition manner.

Finally, to better understand the inhibition mechanism of the conjugate, the effects of L-Apt.4-1c-ASO15nt_{(APP)} and L-Apt.4-1c_5′Hexynyl were compared using L-Apt.4-1c_5′Hexynyl without ASO as negative control. The result indicated that both of them inhibited APP protein level significantly, in which the inhibitory effect of L-Apt.4-1c_5′Hexynyl was weaker than that of L-Apt.4-1c-ASO15nt_{(APP)} (FIG. 3E and 3F), which may be due to the stronger binding affinity of L-Apt.4-1c-ASO15nt_{(APP)} to APP rG4 region than L-Apt.4-1c_5′Hexynyl.

As RNA: DNA hybrid could trigger RNase H cleavage of the RNA, we hypothesized that one possible mechanism underlying the inhibitory effect of L-Apt.4-1c-ASO15nt_{(APP)} may be RNase H-mediated degradation of RNA. To verify this hypothesis, reverse transcription quantitative real-time PCR (RT-qPCR) was performed to measure APP mRNA levels after treatment with L-Apt.4-1c-ASO15nt_{(APP)} or L-Apt.4-1c_5′Hexynyl. It was found that L-Apt.4-1c_5′Hexynyl did not affect APP mRNA levels, whereas L-Apt.4-1c-ASO15nt_{(APP)} significantly reduced APP mRNA levels, suggesting that L-Apt.4-1c_5′Hexynyl inhibited APP translation mainly by stabilizing the rG4 structure, whereas L-Apt.4-1c-ASO15nt_{(APP)} could also recruit RNase H to knock down APP mRNA in addition to translation inhibition. It has been reported that RNase H1 exists in the cytosol of Hela cells. To support this, RNase H1 subcellular distribution was investigated by immunostaining using an RNase H1 antibody together with fluorescently labeled secondary antibody detected by confocal microscopy. The confocal microscopy results showed that RNase H1 was indeed present in the cytosol (data not shown). Then, to examine RNase H effect on the present L-aptamer—ASO conjugate, in vitro RNase H cleavage assay was performed. As expected, the cleavage result showed that the APP rG4 region alone did not induce RNase H cleavage (FIG. 3G, Lane 2), whereas the APP rG4 region & L-Apt.4-1c-ASO15nt_{(APP)} complex was cleaved, and thus generated the cleavage product that can be visualized on denaturing gel (FIG. 3, Lane 4). The cleavage activity was both dosage-and time-dependent (data not shown).

Overall, we demonstrated that L-aptamer—ASO conjugates could inhibit APP protein expression, and the inhibition was mediated by both translation suppression and RNase H-mediated mRNA knockdown.

### Example 4: L-Apt.4-1c-ASO15nt_{(APP)} can Suppress APP rG4—DHX36 Interactions and Control DHX36-Dependent APP rG4-Mediated Gene Expression

In this example, whether DHX36 could bind and unwind APP rG4 was investigated.

To this purpose, the binding test between APP rG4 and DHX36 was first determined by using EMSA, and strong gel shift bands were observed when DHX36 protein was added, confirming that DHX36 protein bound well to APP rG4 region (data not shown).

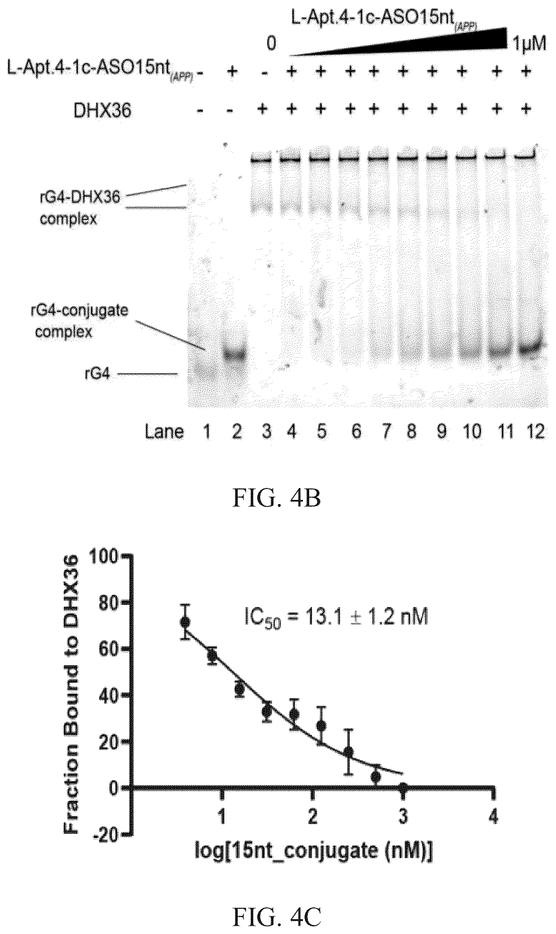

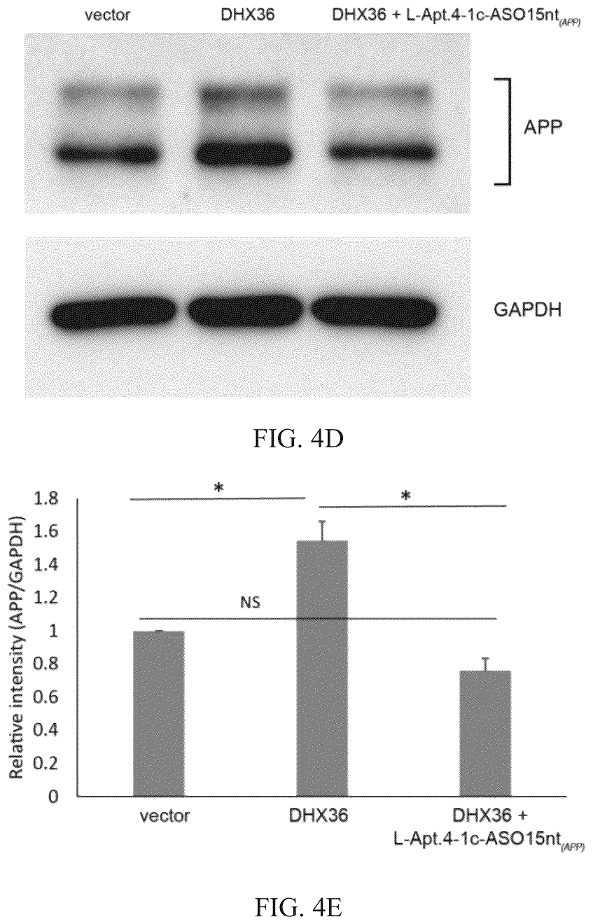

Then, the unwinding ability of DHX36 on APP rG4 was investigated, and a single-stranded RNA (ssRNA) trap oligo (SEQ ID NO: 27) complementary to the unfolded rG4 sequence was designed and used to this purpose. When rG4 was unwound, the ssRNA trap hybridized with the unfolded rG4 sequence and formed a partial duplex to prevent refolding of the rG4 structure, resulting in reduced mobility bands on a native gel by which the process of DHX36 unwinding could be measured. As DHX36 unwinding activity was reported to be ATP-dependent, we also tested its activity using non-hydrolysable analog AMP-PNP as a negative control. To clearly show the shifted bands, a positive marker band was produced by directly hybridizing the ssRNA trap and APP rG4 region sequence with boiling and annealing (FIG. 4A, lane 1). As shown in FIG. 4A, in the presence of the ssRNA trap, DHX36 protein and ATP, an unwound-shift duplex band appeared (FIG. 4A, lane 6). By contrast, no shift bands were found when there was only DHX36 protein (FIG. 4A, lane 4) or DHX36 protein and AMP-PNP (FIG. 4A, lane 5). This suggested that DHX36 could unwind APP rG4 structure in vitro in an ATP-dependent manner.

Whether the conjugate could affect the interaction between APP rG4 region and DHX36protein in vitro was also investigated. To this purpose, a binding competition assay was performed. As APP rG4 region and DHX36 protein could form a complex, and the addition of L-Apt.4-1c-ASO15nt_{(APP)} would disrupt the complex, thereby forming a new APP rG4 region-L-Apt.4-1c-ASO15nt_{(APP)} complex. The EMSA result showed that L-Apt.4-1c-ASO15nt_{(APP)} could efficiently dissociate the binding between APP rG4 region and DHX36 protein, judged from the decreasing of APP rG4 region-DHX36 complex and the rising of APP rG4 region-L-Apt.4-1c-ASO15nt_{(APP)} complex with increased concentration of L-Apt.4-1c-ASO15nt_{(APP)} treatment (FIG. 4B), with a calculated IC₅₀ of 13.1±1.2 nM (FIG. 4C). It is known that the structural domain of the DHX36 protein, the RHAU53 peptide, is responsible for binding to rG4s. To confirm whether L-Apt.4-1c-ASO15nt_{(APP)} disrupted the binding of DHX36 and APP rG4 through the competition with the RHAU53 domain, we examined the disruption effect of the L-Apt.4-1c-ASO15nt_{(APP)} on the binding of the RHAU53 peptide to APP rG4 region. The EMSA result confirmed that the L-Apt.4-1c-ASO15nt_{(APP)} could inhibit their binding efficiently (data not shown).

After confirming the effect of L-Apt.4-1c-ASO15nt_{(APP)} on DHX36 in vitro, whether L-Apt.4-1c-ASO15nt_{(APP)} could also affect DHX36 function in cells was also investigated. The level of DHX36 was overexpressed by transfecting Hela cells with DHX36-pcDNA3.1-3×Flag-C plasmid and pcDNA3.1-3×Flag-C vector was used as negative control. Western blotting results showed that compared with the vector group, DHX36 overexpression elevated APP protein levels through the unwinding of APP rG4. However, when cells were co-transfected with L-Apt.4-1c-ASO15nt_{(APP)} and DHX36 plasmid, APP protein levels were reduced compared with that of DHX36 overexpression group (FIG. 4D and 4E), suggesting that L-Apt.4-1c-ASO15nt_{(APP)} could rescue the DHX36 overexpression effect and control DHX36-dependent APP rG4-mediated gene expression.

Overall, the data indicated that the present L-Apt.4-1c-ASO15nt_{(APP)} conjugate could affect DHX36 function both in vitro and in cells.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An L-form ribonucleic acid (L-RNA) aptamer-antisense oligonucleotide (ASO) conjugate having the structure of formula (I), 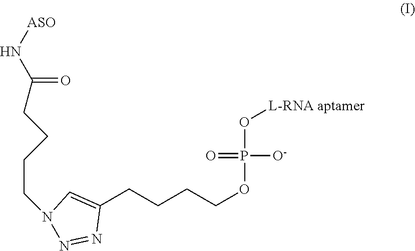
wherein,
the L-RNA aptamer comprises a ribonucleic acid sequence of SEQ ID NO: 1; and
the ASO comprises a deoxyribonucleic acid sequence selected from the group consisting of SEQ ID NOs: 2, 3 or 4.

2. The L-RNA aptamer-ASO conjugate of claim 1, wherein the L-RNA aptamer-ASO conjugate recognizes amyloid precursor protein (APP) 3′-untranslated region (UTR) RNA G-quadruplexes (rG4) structure.

3. The L-RNA aptamer-ASO conjugate of claim 1, further comprising a fluorescein molecular disposed at the 5′-end of the ASO.

4. A method of imaging amyloid precursor protein (APP) rG4 in a cell comprising:
(a) transfecting the cell with a messenger RNA of APP;
(b) permeating the cell;
(c) contacting the permeated cell of step (b) with a cyanine3 (Cy3) labeled APP nucleic acid probe and the L-RNA aptamer-ASO conjugate of claim 3; and
(d) subjecting the product of step (c) to fluorescence microscopy analysis to produce an image of the APP rG4 in the cell.

5. The method of claim 4, wherein in step (b), the cell is permeated by a non-ionic surfactant.

6. The method of claim 5, wherein the non-ionic surfactant is polyethylene glycol octyl phenyl ether.

7. A method of suppressing the expression of amyloid precursor protein (APP) in a cell comprising transfecting the cell with a sufficient amount of the L-RNA aptamer-ASO conjugate of claim 1.

8. The method of claim 7, wherein the transfecting of the cell is achieved by use of lipofectamine.


SEQUENCE LISTING (42 sequences)

SEQ ID NO: 1 - RNA, 25 nt, synthetic construct
  source 1..25: mol_type = other RNA
gccctaaaggtggtggtgggagggc

SEQ ID NO: 2 - DNA, 10 bp, synthetic construct
  source 1..10: mol_type = other DNA
cagcagagca

SEQ ID NO: 3 - DNA, 15 bp, synthetic construct
  source 1..15: mol_type = other DNA
aagaccagcagagca

SEQ ID NO: 4 - DNA, 20 bp, synthetic construct
  source 1..20: mol_type = other DNA
aattgaagaccagcagagca

SEQ ID NO: 5 - DNA, 15 bp, synthetic construct
  source 1..15: mol_type = other DNA
aagaccagcagagca

SEQ ID NO: 6 - RNA, 36 nt, synthetic construct
  source 1..36: mol_type = other RNA
gggagggcggggagggcgcgcggcgatcggacacga

SEQ ID NO: 7 - RNA, 40 nt, synthetic construct
  source 1..40: mol_type = other RNA
gagggagggagggagagggagagagggagaaaacgaaggg

SEQ ID NO: 8 - RNA, 45 nt, synthetic construct
  source 1..45: mol_type = other RNA
gggggccgtggggtgggagctggggcgagaggtgccgttggcccc

SEQ ID NO: 9 - RNA, 38 nt, synthetic construct
  source 1..38: mol_type = other RNA
ggggcgggtggggaggggtgctctgctggtcttcaatt

SEQ ID NO: 10 - RNA, 20 nt, synthetic construct
  source 1..20: mol_type = other RNA
cggggcgggtggggaggggt

SEQ ID NO: 11 - RNA, 20 nt, synthetic construct
  source 1..20: mol_type = other RNA
cgaagcgagtgaagagaagt

SEQ ID NO: 12 - RNA, 38 nt, synthetic construct; identical to SEQ ID NO: 9
  source 1..38: mol_type = other RNA

SEQ ID NO: 13 - RNA, 38 nt, synthetic construct
  source 1..38: mol_type = other RNA
gaagcgagtgaagagaagtgctctgctggtcttcaatt

SEQ ID NO: 14 - DNA, 40 bp, synthetic construct
  source 1..40: mol_type = other DNA
ggagggcgctgggaggaggggctgctgctcgccgctcgcg

SEQ ID NO: 15 - DNA, 37 bp, synthetic construct
  source 1..37: mol_type = other DNA
gggttgcggagggtgggcctgggaggggtggtggcca

SEQ ID NO: 16 - DNA, 44 bp, synthetic construct
  source 1..44: mol_type = other DNA
ttagggttagggttagggttagggttagagttagagttagagtt

SEQ ID NO: 17 - DNA, 43 bp, synthetic construct
  source 1..43: mol_type = other DNA
gggcgcgggaggaagggggcgggagcggggctgtggtgcctgt

SEQ ID NO: 18 - DNA, 40 bp, synthetic construct
  source 1..40: mol_type = other DNA
ggggcgggccgggggcggggtcccggcggggcggagccat

SEQ ID NO: 19 - DNA, 45 bp, synthetic construct
  source 1..45: mol_type = other DNA
gggggccgtggggtgggagctggggcgagaggtgccgttggcccc

SEQ ID NO: 20 - DNA, 40 bp, synthetic construct
  source 1..40: mol_type = other DNA
gagggagggagggagagggagagagggagaaaacgaaggg

SEQ ID NO: 21 - DNA, 36 bp, synthetic construct
  source 1..36: mol_type = other DNA
gggagggcggggagggcgcgcggcgatcggacacga

SEQ ID NO: 22 - RNA, 18 nt, synthetic construct
  source 1..18: mol_type = other RNA
aaaaaaaaaaaaaaaaaa

SEQ ID NO: 23 - RNA, 18 nt, synthetic construct
  source 1..18: mol_type = other RNA
cccccccccccccccccc

SEQ ID NO: 24 - RNA, 18 nt, synthetic construct
  source 1..18: mol_type = other RNA
tttttttttttttttttt

SEQ ID NO: 25 - DNA, 18 bp, synthetic construct
  source 1..18: mol_type = other DNA
cagtacagatctgtactg

SEQ ID NO: 26 - RNA, 18 nt, synthetic construct
  source 1..18: mol_type = other RNA
cagtacagatctgtactg

SEQ ID NO: 27 - RNA, 15 nt, synthetic construct
  source 1..15: mol_type = other RNA
tccccacccgccccg

SEQ ID NO: 28 - DNA, 18 bp, synthetic construct
  source 1..18: mol_type = other DNA
taatacgactcactatag

SEQ ID NO: 29 - DNA, 142 bp, synthetic construct
  source 1..142: mol_type = other DNA
tacaatcatcctgcagaaaattgttttggagaattcttggtaattgaagaccagcagagcacccctccccacccgccccgtaaaagtgcttacaatgaacagggattcttttctttatcaaagactatagtgagtcgtatta

SEQ ID NO: 30 - DNA, 109 bp, synthetic construct
  source 1..109: mol_type = other DNA
tacaatcatcctgcagaaaattgttttggagaattcttggtaattggtaaaagtgcttacaatgaacagggattcttttctttatcaaagactatagtgagtcgtatta

SEQ ID NO: 31 - DNA, 140 bp, synthetic construct
  source 1..140: mol_type = other DNA
tgcccgcccgctgccgtcgctactcccgcctcctcccgccatcgtgtccgatcgccgcgcgccctccccgccctcccggccgggccgcttcctcggctgtgacgccgctgggtcacgcacgactatagtgagtcgtatta

SEQ ID NO: 32 - DNA, 142 bp, synthetic construct
  source 1..142: mol_type = other DNA
taagatcatagtgaactgtgcttcaatggatggacgagctccccttcgttttctccctctctccctctccctccctccctcgtttcctttcaaaaaaaagtcctccgggtgggtaaggagcctgctatagtgagtcgtatta

SEQ ID NO: 33 - DNA, 25 bp, synthetic construct
  source 1..25: mol_type = other DNA
tacaatcatcctgcagaaaattgtt

SEQ ID NO: 34 - DNA, 25 bp, synthetic construct
  source 1..25: mol_type = other DNA
tgcccgcccgctgccgtcgctactc

SEQ ID NO: 35 - DNA, 25 bp, synthetic construct
  source 1..25: mol_type = other DNA
taagatcatagtgaactgtgcttca

SEQ ID NO: 36 - DNA, 21 bp, synthetic construct
  source 1..21: mol_type = other DNA
ggagcgagatccctccaaaat

SEQ ID NO: 37 - DNA, 23 bp, synthetic construct
  source 1..23: mol_type = other DNA
ggctgttgtcatacttctcatgg

SEQ ID NO: 38 - DNA, 20 bp, synthetic construct
  source 1..20: mol_type = other DNA
gtgctctgctggtcttcaat

SEQ ID NO: 39 - DNA, 20 bp, synthetic construct
  source 1..20: mol_type = other DNA
gaaaagtcttgcccggggtt

SEQ ID NO: 40 - DNA, 2982 bp, Homo sp.
  source 1..2982: mol_type = genomic DNA
atgagttatgactaccatcagaactggggccgtgatgggggtccccgcagctccggtgggggctatggaggggggccagcagggggtcatggaggtaaccgaggctccggaggaggcggcggcggcggagggggtggtcgaggcggcaggggccggcatcccgggcacctgaaaggccgcgaaatcggcatgtggtacgcgaaaaaacaggggcagaagaacaaggaagcggagaggcaagagagagctgtagtacacatggatgaacgacgagaagaacaaattgtacagttactgaattctgttcaagcgaagaatgataaagagtcagaagcacagatatcctggtttgctcctgaggatcatggatacggtactgaagtttctactaagaacacaccatgctcagagaacaaacttgacatccaggaaaagaagttgataaatcaagaaaaaaaaatgtttagaatcaggaacagatcatatattgaccgagattctgagtatctcttgcaagaaaatgaaccagatggaactttagaccaaaaattattggaagatttacaaaagaaaaaaaatgaccttcggtatattgaaatgcagcatttcagagaaaagctgccttcgtatggaatgcaaaaggaattggtaaatttaattgataaccatcaggtaacagtaataagtggtgaaactggttgtggcaaaaccactcaagttactcagttcattttggataactacattgaaagaggaaaaggatctgcttgcagaatagtttgtactcagccaagaagaattagtgccatttcagttgcggaaagagtagctgcagaaagggcagaatcttgtggcagtggtaatagtactggatatcaaattcgtctccagagtcggttgccaaggaaacagggttctatcttatactgtacaacaggaatcatccttcagtggctccagtcagacccgtatttgtccagtgttagtcatatcgtacttgatgaaatccatgaaagaaatctgcagtcagatgttttaatgactgttgttaaagaccttctcaattttcgatctgacttgaaagtaatattgatgagtgcaacattgaatgcagaaaagttttcagaatattttggtaactgtccaatgatacatatacctggttttacctttccggttgtggaatatcttttggaagatgtaattgaaaaaataaggtatgttccagaacaaaaagaacacagatcccagtttaagaggggtttcatgcaagggcatgtaaatagacaagaaaaagaagaaaaagaagcaatatataaagaacgttggccagattatgtaagggaactgcgaagaaggtattctgcaagtactgtagatgttatagaaatgatggaggatgataaagttgatctgaatttgattgttgccctcatccgatacattgttttggaagaagaggatggtgcgatactggtctttctgccaggctgggacaatatcagcactttacatgatctcttgatgtcacaagtaatgtttaaatcagttaaccagacacaggtgtttaaaagaacccctcctggtgttcggaaaatagtaattgctaccaacattgcggagactagcattaccatagatgatgtcgtttatgtgatagatggaggaaaaataaaagagacacattttgatactcagaacaatatcagtacaatgtccgctgagtgggttagtaaagctaatgccaaacagagaaaaggtcgagctggaagagttcaacctggtcattgctatcatctgtataatggtcttagagcaagtcttctagatgactatcaactgccagaaattttgagaactcctttggaagaactttgtttacaaataaagattttaaggctaggtggaattgcttattttctgagtagattaatggacccaccatcaaatgaggcagtgttactctccataagacacctgatggagctgaacgctttggataaacaagaagaattgacacctcttggagtccacttggcacgattacccgttgagccacatattggaaaaatgattctttttggagcactgttctgctgcttagacccagtactcactattgctgctagtctcagtttcaaagatccatttgtcattccactgggaaaagaaaagattgcagatgcaagaagaaaggaattggcaaaggatactagaagtgatcacttaacagttgtgaatgcgtttgagggctgggaagaggctaggcgacgtggtttcagatacgaaaaggactattgctgggaatattttctgtcttcaaacacactgcagatgctgcataacatgaaaggacagtttgctgagcatcttcttggagctggatttgtaagcagtagaaatcctaaagatccagaatctaatataaattcagataatgagaagataattaaagctgtcatctgtgctggtttatatcccaaagttgctaaaattcgactaaatttgggtaaaaaaagaaaaatggtaaaagtttacacaaaaaccgatggcctggttgctgttcatcctaaatctgttaatgtggagcaaacagactttcactacaactggcttatctatcacctaaagatgagaacaagcagtatatacttgtatgactgcacagaggtttccccatactgtctcttgttttttggaggtgacatttccatccagaaggataacgatcaggaaactattgctgtagatgagtggattgtatttcagtctccagcaagaattgcccatcttgttaaggaattaagaaaggaactagatattcttctgcaagagaagattgaaagtcctcatcctgtagactggaatgacactaaatccagagactgtgcagtactgtcagctattatagacttgatcaaaacacaggaaaaggcaactcccaggaactttccgccacgattccaggatggatattacagc

SEQ ID NO: 41 - protein, 1039 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRCQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSDKFLIIPLHSLMPTVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYSTRTRPLEQKLISEEDLAANDILDYKDDDDKV

SEQ ID NO: 42 - protein, 53 aa, synthetic construct
HPGHLKGREIGMWYAKKQGQKNKEAERQERAVVHMDERREEQIVQLLNSVQAK